(19) United States
(12) Patent Application Publication
(10) Pub. No.: US 2019/0038224 A1
(43) Pub. Date: Feb. 7, 2019

(54) WEARABLE DEVICES HAVING PRESSURE ACTIVATED BIOMETRIC MONITORING SYSTEMS AND RELATED METHODS

(71) Applicant: Intel Corporation, Santa Clara, CA (US)

(72) Inventors: Zhaonian Zhang, Sunnyvale, CA (US); Robert Lau, Danville, CA (US)

(21) Appl. No.: 15/668,515

(22) Filed: Aug. 3, 2017

(51) Int. Cl. A61B 5/00; H04W 52/02

(52) U.S. Cl. CPC A61B 5/6824; A61B 5/021; A61B 5/6831; H04W 52/02

(57) ABSTRACT

Wearable devices having pressure activated biometric monitoring systems and related methods are disclosed. An example wearable device includes a housing including a biometric sensor to detect physiological information of a user. The wearable device includes a circuit to electrically couple the biometric sensor and a power source of the wearable device. A spring contact is positioned adjacent the biometric sensor and is to be oriented toward a user. The spring contact is movably coupled relative to the housing. The spring contact is to close the circuit to activate the biometric sensor when the wearable device is strapped to the user and the spring contact engages the user. The spring contact is to open the circuit to deactivate the biometric sensor when the wearable device is removed from the user to reduce energy drain of the power source by the biometric sensor.

### FIELD OF THE DISCLOSURE

This disclosure relates generally to wearable devices, and, more particularly, to wearable devices having pressure activated optical sensor systems.

### BACKGROUND

Wearable biometric monitoring devices employ optical sensor systems to detect and/or measure physiological metrics or information from a subject wearing the monitoring device.

### BRIEF DESCRIPTION OF THE DRAWINGS

FIG. 1 is a schematic illustration of an example wearable device implemented in accordance with the teachings of this disclosure.

FIG. 2A is a front, perspective view of an example implementation of the example wearable device of FIG. 1 strapped to a body of a user.

FIG. 2B is a rear, perspective view of the example wearable device of FIG. 2A removed from the user.

FIG. 2C is a side view of the example wearable device of FIGS. 2A-2B.

FIG. 3A is a schematic illustration of an example pressure actuator disclosed herein that may implement the example wearable device of FIG. 1 shown in a first position.

FIG. 3B is a schematic illustrated of the example pressure actuator of FIG. 3A shown in a second position.

FIG. 4 is a schematic illustration of another example wearable device disclosed herein.

FIG. 5 is a block diagram of an example biometric sensor activator of the example wearable device of FIG. 4.

FIG. 6 is an example implementation of the example wearable device of FIGS. 5-6.

FIG. 7 is a flowchart representative of example machine readable instructions which may be executed to implement the example wearable device of FIGS. 4-6.

FIG. 8 is a schematic illustration of an example processor platform that may execute the instructions of FIG. 7 to implement the example wearable device of FIGS. 4-6.

The figures are not to scale. Instead, to clarify multiple layers and regions, the thickness of the layers may be enlarged in the drawings. Wherever possible, the same reference numbers will be used throughout the drawing(s) and accompanying written description to refer to the same or like parts. As used in this patent, stating that any part (e.g., a layer, film, area, or plate) is in any way positioned on (e.g., positioned on, located on, disposed on, or formed on, etc.) another part, indicates that the referenced part is either in contact with the other part, or that the referenced part is above the other part with one or more intermediate part(s) located therebetween. Stating that any part is in contact with another part means that there is no intermediate part between the two parts. Stating that a part is coupled or connected to another part indicates that the parts are jointed directly or through one or more intervening parts. Thus, physical contact is not required for two parts to be coupled or connected.

### DETAILED DESCRIPTION

Wearable devices employ biometric monitoring systems to detect biological or physiological information of a user. In some examples, wearable devices employ intelligent platforms (e.g., a processor, a System on a Chip (SoC), etc.) that provide the capability to continuously and unobtrusively monitor physiological metrics. For example, wrist worn optical heart rate monitors (OHRMs) have become more prevalent in wearable devices to monitor health activity of a user. Example heart rate monitors include one or more light sources (e.g., light emitting diodes) and a photo sensor (e.g., a photodiode). For example, the LEDs emit visible light that is reflected by the skin. Blood circulation within the body can modulate the strength of the reflected light, which is captured by the photo sensor and translated it into heart rate after signal processing.

To provide power to the optical sensor system, wearable devices typically employ a power source such as, for example, a battery (e.g., a rechargeable battery). Some known wearable devices provide continuous power to the biometric sensor (e.g., OHRM), even when the wearable device is not being worn by user. However, when the wearable device is not being worn by a user, the biometric sensor (e.g., OHRM) may continue to draw and/or consume power from the power source, thereby depleting the stored energy of the power source and reducing the operating life of the power source and/or the wearable device. In some examples, providing continuous power to the biometric monitoring systems may increase (e.g., elevate) an operating temperature of the wearable device, which may cause damage to electrical components (e.g., a processor) of the wearable device.

To provide power to the biometric monitoring system when a wearable device is worn by a user, some known devices employ a proximity sensor to detect the presence of a user (e.g., when the wearable device is positioned against an arm of the user). Thus, the wearable device provides power to the biometric sensor (e.g. OHRM) from the power source (e.g., only) upon detection of the wearable device being coupled to a user (e.g., an arm of a user). However, such approach can result in false positives when the wearable device is positioned on a flat surface such as, for example, an office desk.

Example wearable devices disclosed herein employ pressure activated biometric monitoring systems. More specifically, example wearable devices disclosed herein rely on pressure to control biometric monitoring system functionality. For example, example wearable devices disclosed herein employ pressure-activated switches to enable/disable the biometric monitoring systems (e.g., OHRMs). More specifically, example biometric monitoring systems of example wearable devices disclosed herein activate when the wearable device is worn by a user for its intended use.

In this manner, example wearable devices deactivate biometric monitoring system functionality when the wearable device is positioned on a flat surface such as, for example, an office desk, a dresser, a table, etc. In some examples, if the device is not being worn and/or is not on a person's body (e.g., a wrist), the wearable device electrically decouples the biometric monitoring system from a power source to reduce depletion and/or consumption of energy of the power source by the biometric monitoring device when the device is not in use or worn by a user. However, if the device is in use (e.g., worn and/or attached to one's wrist), wearable devices disclosed herein electrically couple the biometric monitoring system and the power source to activate biometric monitoring system functionality and enable the biometric monitoring system to measure and/or otherwise obtain physiological information of the user.

To provide a pressure activated monitoring system, example wearable devices disclosed herein provide a signal generator that closes a circuit (e.g., via switch) to activate a biometric sensor (e.g., a light source and/or photo sensor) of a biometric monitoring system (e.g., an optical heart rate monitor (OHRM)) when the wearable device is mounted, strapped, and/or otherwise coupled to a body portion (e.g., a wrist) of a user and opens the circuit (e.g., shorts the circuit) to reduce (e.g., stop or prevent) power drain (e.g., battery drain) by the biometric sensor (e.g., the light source and/or photo sensor) when the wearable device is removed from the body portion of the user (e.g., the device is not strapped to the user). A signal generator disclosed herein may include, for example, a spring contact, a switch (e.g., a mechanical switch, an electrical switch, etc.), a spring-loaded contact (e.g., a spring-loaded button), a pressure sensor, a transistor, a dome-switch, a piezoelectric sensor and/or any other device that generates a signal in response to a force or pressure generated when the wearable device is strapped or coupled to a user.

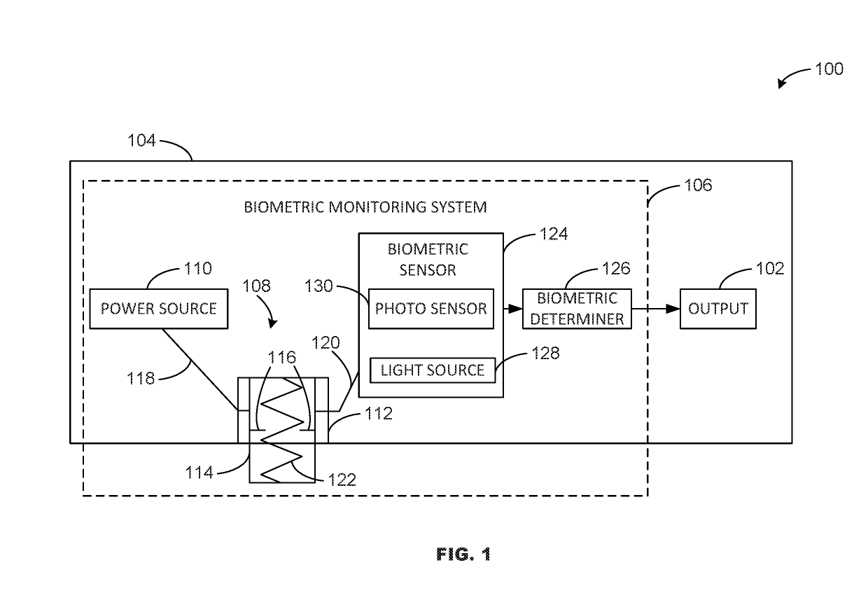

FIG. 1 is a schematic diagram of an example wearable device 100 constructed in accordance with the teachings of this disclosure. The wearable device of the illustrated example may be a watch, a bracelet, a fashionable device, a smart electronic device (e.g., electronic devices with microcontrollers, a smart watch, etc.), and/or any other device including a processor and/or communication capability that can be worn on a body of a user. For example, the wearable device 100 of the illustrated example may be worn around a person's wrist, finger, arm, leg, ankle, torso, head, and/or any other body portion. The wearable device of the illustrated example can output information to a user via an output 102 (e.g., a display, an LED display) and/or may communicate via wired or wireless communication with other devices (e.g., external devices).

The wearable device 100 of the illustrated example includes a housing 104 having a biometric monitoring system 106 to detect a biometric or physiological characteristic(s) or information of a user. To enable biometric monitoring functionality, the biometric monitoring system 106 of the illustrated includes a circuit 108 to electrically couple the biometric monitoring system 106 to a power source 110 that provides power (e.g., DC current, DC voltage) to the biometric monitoring system 106. The power source 110 of the illustrated example may be a lithium-ion battery (e.g., a rechargeable battery). In some example, the power source 110 is a dedicated power source of the biometric monitoring system 106. In other examples, the power source 110 may power other electrical component(s) of the wearable device 100. For example, the power source 110 may power a processor (e.g., a system-on-chip (SoC)), a display, a camera and/or another other electrical component(s) of the wearable device 100.

To enable and/or disable the biometric monitoring system functionality, the wearable device 100 of the illustrated example includes a signal generator or switch 112. The switch 112 of the illustrated example electrically couples and decouples the power source 110 and the biometric monitoring system 106. More specifically, the switch 112 of the illustrated example operates based on a pressure or force such that the switch 112 closes the circuit 108 to activate biometric monitoring system functionality when the wearable device 100 is strapped to the user and opens the circuit 108 (e.g., shorts the circuit 108) to deactivate the biometric monitoring system functionality when the wearable device 100 is not strapped to (e.g., not worn or is removed from) the user to reduce (e.g., decrease or minimize) draining of the power source 110 by the biometric monitoring system 106 when the wearable device 100 is not worn by a user.

To open and close the circuit 108 based on a pressure or force, the switch 112 of the illustrated example is movable relative to the housing 104 between a first position (e.g., an activated position) and a second position (e.g., a non-activated position). For example, FIG. 1 illustrates the switch 112 in the second position relative to the housing 104. In the first position, for example, the switch 112 of the illustrated example enables biometric monitoring functionality of the biometric monitoring system 106. In the second position, for example, the switch 112 of the illustrated example disables biometric monitoring functionality of the biometric monitoring system 106.

To move the switch 112 between the first position and the second position, the switch 112 of the illustrated example includes a spring contact 114 movable relative to the housing 104. To electrically couple the power source 110 and the biometric monitoring system 106 via the circuit 108, the spring contact 114 of the illustrated example includes an electrically conductive contact 116 (e.g., an electrically conductive trace) that electrically couples a conductive trace 118 of the power source 110 and a conductive trace 120 of the biometric monitoring system 106 when the spring contact 114 is in the first position. To electrically decouple the power source 110 and the biometric monitoring system 106 via the circuit 108 as shown, for example, in FIG. 1, the switch 112 of the illustrated example electrically decouples or interrupts the connection between the conductive trace 118 of the power source 110 and the conductive trace 120 of the biometric monitoring system 106 when the spring contact 114 is in the second position and positioned away from the conductive traces 118 and 120. The circuit 108 and/or the biometric monitoring system 106 of the illustrated example may be formed via an integrated circuit (e.g., a printed circuit board) positioned in the housing 104. In some examples, at least a portion of the conductive traces 118 and 120 may be formed (e.g., embedded) in the housing 104 of the wearable device 100. In some examples, the switch 112 provides means for activating the biometric monitoring system 106. In some examples, the spring contact 114 provides means for electrically coupling and electrically decoupling the power source 110 and the biometric monitoring system 106.

Additionally, to enable biometric monitoring system functionality when the wearable device 100 is strapped or clamped to a user and/or to prevent biometric monitoring system functionality when the wearable device 100 is not strapped to or worn by a user, the switch 112 and/or the spring contact 114 of the illustrated example includes a biasing element 122. The biasing element 122 of the illustrated example urges the spring contact 114 and/or the switch 112 toward the second position (e.g., a position that opens the circuit 108). Thus, the switch 112 of the illustrated example is a normally open switch.

The biasing element 122 of the illustrated example provides a biasing force that allows the spring contact 114 to move to the first position to electrically couple the power source 110 and the biometric monitoring system 106 when the housing 104 of the wearable device is strapped or coupled to a user. In particular, a spring force provided by the biasing element 122 is such that the spring contact 114 moves to the first position when the housing 104 of the wearable device 100 is strapped or clapped to a body portion (e.g., a wrist, an angle, an arm, a leg, a head, a torso, etc.) of a user. To strap or clamp the housing 104 to around a body portion (e.g., a wrist, a torso, a finger, etc.) of a user, the housing 104 may include one or more fastener(s) (e.g., a clasp, a strap, a connector, a latch, etc.).

In other words, a clamping force generated when coupling the housing 104 to the user is required to overcome the spring force of the biasing element 122 to enable the switch 112 to move to the first position and close the circuit 108. Thus, the biasing element 122 of the illustrated example enables activation of the biometric monitoring system 106 based on a force and/or pressure applied to the switch 112 as result of the wearable device 100 being coupled (e.g., attached or strapped) to a user. Additionally, the biasing element 122 of the illustrated example prevents the spring contact 114 from moving to the first position when the wearable device 100 is not strapped or clapped to a user (e.g., not worn by a user). In some such examples, when the housing 104 is positioned on a surface (e.g., a flat surface, a night stand, a table and/or any other surface) and/or when the housing 104 is not clamped and/or worn by a user, the biasing element 122 biases the spring contact 114 toward the second position open the circuit 108 and restricts the switch 112 from moving to the first position.

The biometric monitoring system 106 of the illustrated example includes a biometric sensor 124 and a biometric determiner 126. The biometric monitoring system 106 of the illustrated example employs, for example, photopleythsmogram (PPG) techniques to detect or determine biometric information or characteristic(s) of a user. To detect a PPG signal from a user, the biometric sensor 124 of the illustrated example is an optical sensor that includes a light source 128 (e.g., the light emitting diode (LED)) and a light detector or photo sensor 130 (e.g., the photodiode). In some examples, the light detector 130 has a spectral sensitivity that spans at least from green to red, or at least spanning the spectral bandwidths of the two different color light sources (e.g. LEDs). The biometric sensor 124 of the illustrated example may include one or more light source(s) and/or one or more light detector(s).

The biometric sensor 124 of the illustrated example may be an optical heart rate monitor that measures heart rate of a user wearing the wearable device 100. To detect a heart rate, for example, the optical heart rate monitor employs the biometric sensor 124 to detect pulses passing through small blood vessels near the skin of a user. In other examples, the biometric monitoring system 106 of the illustrated example can be used to monitor, detect and/or determine other physiological information including, but not limited to blood pressure, oxygen saturation, blood glucose levels, pulse rate, cardiac information and/or another physiological information of a user.

In the illustrated example, the conductive trace 120 is electrically coupled to the light source 128 of the biometric sensor 124. Thus, when the switch 112 moves to the first position, the power source 110 provides power to the light source 128 so that the light source 128 emits light onto a user's skin. Thus, when the switch 112 is in the second position and/or opens the circuit 108, power (e.g., electrical current or voltage) from the power source 110 to the light source 128 is interrupted. In some examples, the photo sensor 130 and/or the biometric determiner 126 may be electrically coupled to the light source 128 in series such that power interruption to the light source 128 also results in power interruption to the photo sensor 130 and/or the biometric determiner 126 when switch 112 is in the second position (e.g. opens the circuit 108). In other examples, the switch 112 may be electrically coupled to the light source 128, the photo sensor 130 and/or the biometric determiner 126 (e.g., in parallel).

When the housing 104 of the illustrated example is clamped to a body of a user, the light source 128 and the photo sensor 130 are positioned in proximity (e.g., adjacent the user's skin and close to each other) as the light source 128 emits light into the user's skin. As the light penetrates the skin (e.g., a limited skin depth), a portion of light is reflected or backscattered from components like tissue, bones, veins, arteries and the like. The photo sensor 130 of the illustrated example detects the backscattered or reflected light and communicates the reflected light signal to the biometric determiner 126. For example, the photo sensor 130 receives the reflected light and converts it to a current (e.g., AC or DC current). In some examples, the signal from the photo sensor 130 may be voltage (e.g. DC Voltage). A profile of the signal resulting from the backscattered light represents a photoplethysmography or PGG signal.

To determine and/or obtain physiological information of the user, the biometric determiner 126 of the illustrated example processes the signals (e.g., PGG signals) from the photo sensor 130. In some examples, the biometric determiner 126 may include a filter to limit noise bandwidth of the photo sensor 130. In some examples, the biometric determiner 126 of the illustrated example converts the signals from the photo sensor 130 to a differential voltage to determine the physiological information. In some examples, the biometric determiner 126 employs a signal processing algorithm a matrix (e.g. a look-up table) to extract or determine the physiological information (e.g., heart rate) based on the signal(s) from the photo sensor 130. In some examples, the biometric monitoring system 106, the biometric sensor 124, the biometric determiner 126, the light source 128 and/or the photo sensor 130 provides means for sensing physiological information of a user.

The biometric determiner 126 of the illustrated example presents the determined physiological information via the output 102 of the wearable device 100. The output 102 of the illustrated example may be, for example, a user interface, a display, an output port (e.g., a USB port), an antenna (e.g., a bluetooth antenna) and/or any another output to communicate and/or present the physiological information (e.g., heart rate) to the user wearing the wearable device 100 and/or to other external devices (e.g., a computer, a server, the internet, etc.).

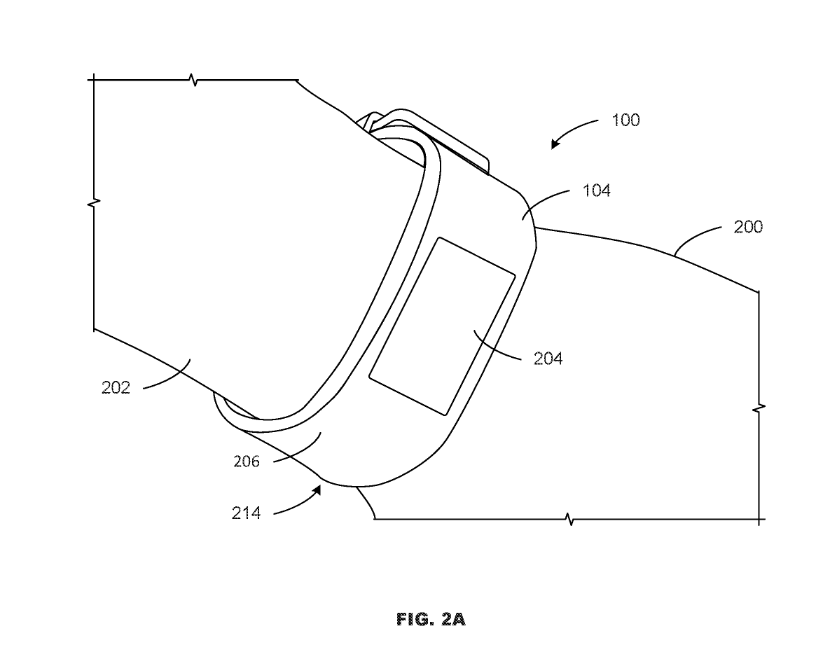

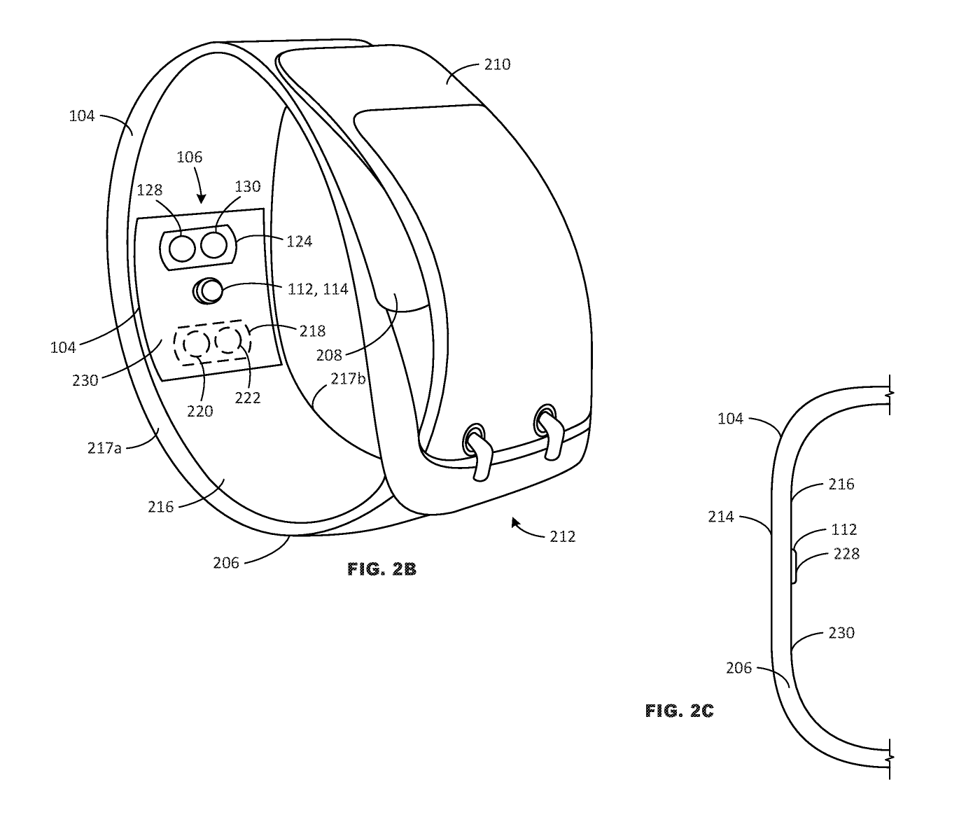

FIG. 2A illustrates an example implementation of the example wearable device 100 of FIG. 1 as a wrist-worn device. FIG. 2B is a perspective, rear view of the example wearable device 100 of FIG. 2A. FIG. 2C is a partial side view of the example wearable device 100 of FIGS. 2A and 2B. The wearable device 100 of FIGS. 2A-2C may be a bracelet, a watch, a strap, and/or any other wrist-worn device configured to wrap around and/or clamp against an arm or wrist 202 of a user 200. The wearable device 100 of the illustrated example includes a display 204 (e.g., a touch screen display) to present information (e.g. physiological information) to a user of the wearable device 100.

The wearable device 100 of the illustrated example includes the housing 104 to house the biometric monitoring system 106 and the switch 112. The housing 104 of the illustrated example includes a strap 206 having a first end 208 and a second end 210 opposite the first end 208. The first end 208 overlaps the second end 210 when the housing 104 is coupled to the user 200.

To couple or clamp the housing 104 to the wrist 202 of the user 200, the housing 104 and/or the strap 206 of the illustrated example includes a fastener or connector 212. In particular, the connector 212 of the illustrated example couples the first end 208 of the strap 206 and the second end 210 of the strap 206. Additionally, the connector 212 causes the strap 206 and/or housing 104 of the illustrated example to clamp (e.g., securely or tightly) onto the user 200 to prevent or restrict the wearable device 100 from falling off the wrist 202 and/or rotating relative to the wrist 202 of the user 200 during use. The connector 212 of the illustrated example is a buckle. However, in some examples, the connector 212 may include any type of connector such as, for example, a latch, a folding clasp, a butterfly clasp, a magnetic coupler, velcro, and/or any other fastener(s) to couple the first end 208 and the second end 210 and/or to enable the wearable device 100 to couple (e.g., clamp or strap) to a person's wrist and/or other part of the body. In the illustrated example, the strap 206 is coupled to the housing 104 and is positionable between a closed position to clamp the housing 104 against the body (e.g., the wrist 202) of the user 200 and an open position to release the housing 104 from the body of the user 200. In some examples, the strap 206 and/or the connector 212 provide means for clamping the housing 104 (e.g., the biometric sensor 124 and/or the switch 112) to a body of a user.

The example wearable device 100 of the illustrated example defines a first side 214 and a second side 216 opposite the first side 214. The first side 214 of the example housing 104 of the illustrated example is oriented away from the user's skin and the and the second side 216 of the housing 104 of the illustrated example is oriented toward the user's skin. In particular, the second side 216 of the housing of the illustrated example is to engage the skin of the user when the wearable device 100 is strapped to the user's wrist 202.

The housing 104 of the illustrated example houses the biometric monitoring system 106 and the switch 112. In particular, the biometric sensor 124 of the biometric monitoring system 106 is positioned on the second side 216 of the housing 104 and oriented to engage (e.g., directly contact) the user's skin when the wearable device 100 is strapped or clamped to the user 200. In the illustrated example, the biometric sensor 124 of the illustrated example includes a first optical sensor 218a having the light source 128 and the corresponding photo sensor 130. In some examples, the wearable device 100 and/or the biometric sensor 124 of the illustrated example may include a second optical sensor 218b including a second light source 220 (e.g., a LED) and a corresponding second photo sensor 222 (e.g., a photodiode). However, in some examples, the biometric sensor 124 may include only one light source (e.g., the light source 128) and a plurality of light detectors (e.g., the photo sensors 130 and 222) or, alternatively, a plurality of light sources (e.g., the light sources 128 and 220) and only one light detector (e.g., the photo sensor 130).

Additionally, the switch 112 of the illustrated example is positioned adjacent the biometric sensor 124. In the illustrated example, the switch 112 is positioned adjacent the optical sensor 218a. For example, the switch 112 may be centrally located between lateral edges 217a and 217b of the housing 104. In some examples, the switch 112 may be positioned between the biometric sensor 124 and the second optical sensor 218b. The switch 112 of the illustrated example is a spring contact 114 (e.g., a spring-loaded button). More specifically, the spring contact 114 of the illustrated example moves relative to the housing 104 between the first position to activate the first and second optical sensors 218a and 218b and the second position to deactivate the first and second optical sensors 218a and 218b. More specifically, in the second position, the spring contact 114 of the illustrated example at least partially protrudes from the second side 216 of the housing 104. In some examples, in the first position, an outer surface 228 of the spring contact 114 is flush with an outer surface 230 of the second side 216 of the housing 104. For example, the outer surface 228 of the spring contact 114 may be evenly aligned with the outer surface 230 of the second side 216 of the housing 104 when the spring contact 114 is in the first position. In some examples, the outer surface 228 of the spring contact 114 protrudes a greater distance from the outer surface 230 of the second side 216 of the housing 104 when the spring contact 114 is in the second position than when the spring contact 114 is in the first position.

A clamping force around the wrist 202 of the user 200 that is generated when the connector 212 fastens the first end 208 and the second end 210 causes the switch 112 or the spring contact 114 to move to the first position to activate the biometric monitoring system 106 (e.g., the first and second optical sensors 218a and 218b). As the first end 208 of the strap 206 is coupled to the second end 210 of the housing 104 via the connector 212, the connector 212 causes a force or pressure to be applied to the switch 112 against the user's body, thereby causing the spring contact 114 to move against the spring force of the biasing element 122 (e.g., FIG. 1) to move the switch 112 from the second position to the first position and close the circuit 108. Thus, the switch 112 is a pressure-activated switch that moves to the first position (e.g., only) when the wearable device 100 is worn by a user and the connector 212 is in a clamping or closed position.

On the contrary, if the connector 212 is not in the closed or clamping position to establish a clamping force against the wrist 202 of the user 200, the switch 112 remains in the second position, thereby deactivating the biometric sensor 124. For example, if the housing 104 is positioned on a flat surface such as a table (e.g., with the first end 208 and the second end 210 are in a decoupled condition), the biasing element 122 urges the spring contact 114 to the second position and restricts the switch 112 from moving to the first position. In other words, the spring force provided by the biasing element 122 is greater than (e.g., supports) a weight of the housing 104 when the wearable device 100 is positioned on the flat surface and/or the wearable device 100 is in the strapped to the user 200 (e.g., the connector 212 is in an open position). In some examples, the switch 112 (e.g., the spring contact 114) prevents or deters laying the second side 216 of the housing 104 of the wearable device 100 on a surface (e.g., a table or desk). In some such examples, the wearable device 100 (e.g., the housing 104) can be positioned on a surface (e.g., a flat surface, a desk, etc.) by positioning the housing 104 on a side surface (e.g. a surface perpendicular or non-parallel relative to the second side 216), or positioning the first side 214 of the housing 104 to lay on the surface (e.g., a flat surface or desk).

In some examples, having the first end 208 and the second end 210 coupled and wrapped around the wrist 202 of the user 200 is indicative of the wearable device 100 being worn by an individual. In other examples, having the first end 208 and the second end 210 not coupled (e.g., even when worn by the user 200) and/or are otherwise not immediately adjacent to one another is indicative of the wearable device 100 not being worn by an individual. In examples where the first end 208 and the second end 210 include a buckle to enable the first end 208 and the second end 210 to be coupled, the first end 208 and the second end 210 may be considered not coupled if a pin of the buckle is not in contact with a remainder of the buckle. In some examples, the switch 112 may be movably coupled to at least one of the housing 104, the strap 206, the first end 208, the second end 210 and/or any other portion of the wearable device 100 that engages the skin of the user.

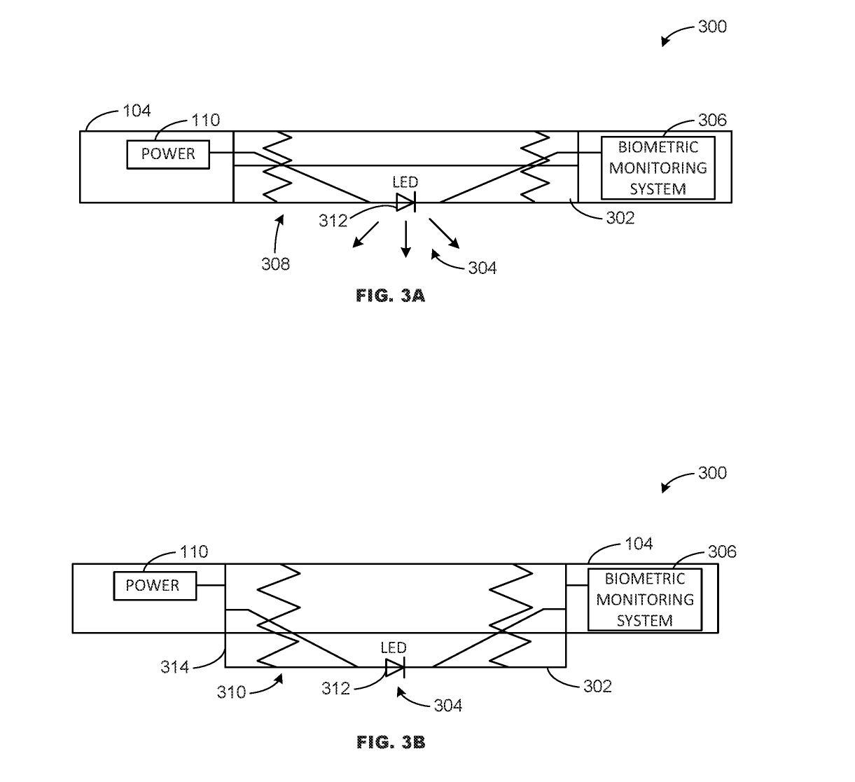

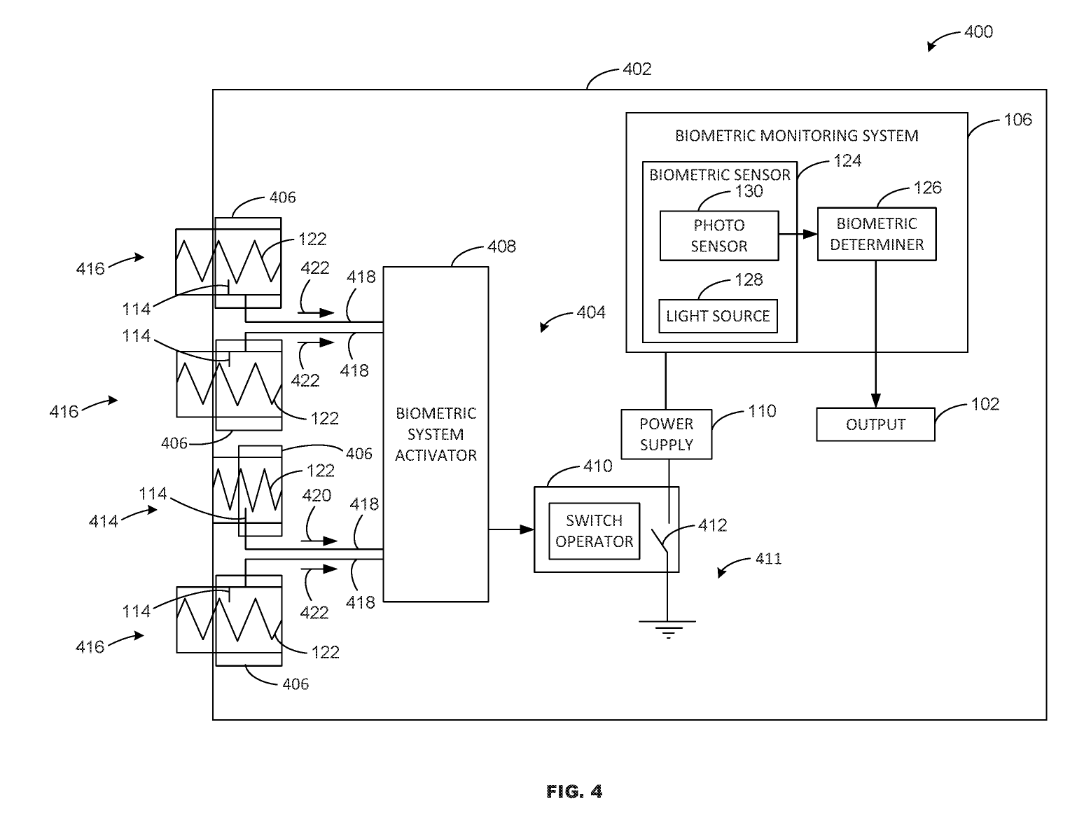

FIGS. 3A and 3B illustrate another example signal generator or switch 300 that may be used to implement the wearable device 100 of FIGS. 1 and 2A-2C. For example, the switch 300 of the illustrated example may be used in place of the switch 112 of FIGS. 1 and 2A-2C. The switch 300 of the illustrated example includes a spring contact 302 (e.g., a spring-loaded button). The spring contact 302 of the illustrated example is movable relative to the housing 104 of the wearable device 100 between a first position 308 to close the circuit 108 as shown in FIG. 3A and a second position 310 to open the circuit 108 as shown in FIG. 3B.

Unlike the switch 112 of FIGS. 1 and 2A-2C, at least a portion of the biometric sensor 304 of the illustrated example is formed or positioned with the switch 300 and/or the spring contact 302. For example, the biometric sensor 304 of the illustrated example includes a light source 312 (e.g. a LED) that is formed or positioned in a body 314 of the switch 300 (e.g. a spring-loaded button). In some examples, a photo sensor (e.g., the photo sensor 130 of FIGS. 1 and 2A-2C) may be positioned and/or formed with the body 314 of the switch 300. Thus, in some examples, the light source 312 and/or the photo sensor 130 may be formed with the switch 300. The biometric sensor 304 of the illustrated example is positioned on a surface 316 of the switch 300 that is to engage or contact (e.g., directly engage) a body portion of the user.

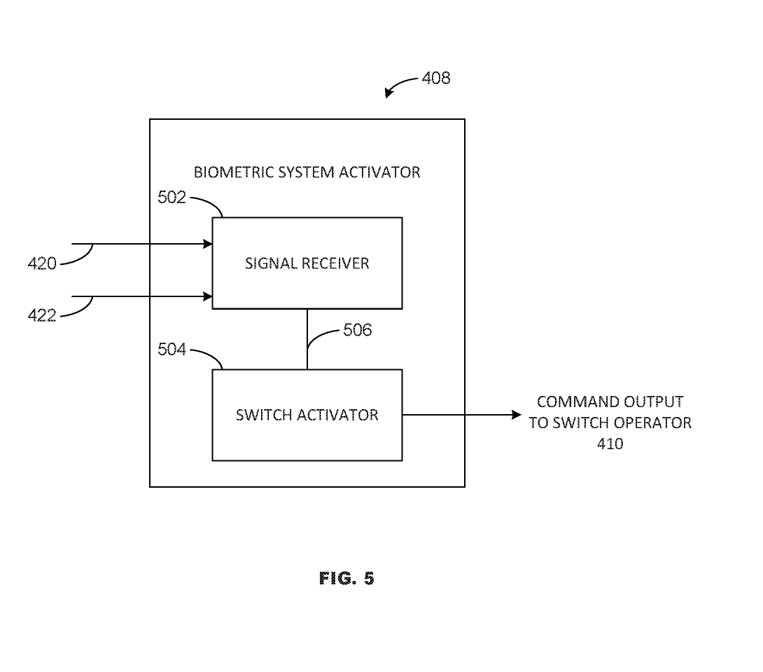

FIG. 4 is schematic illustration of another example wearable device 400 disclosed herein. Those components of the example wearable device 400 that are substantially similar or identical to the components of the example wearable device 100 described above with reference to FIGS. 1 and 2A-2C will not be described in detail again below. Instead, the interested reader is referred to the above corresponding descriptions. To facilitate this process, similar reference numbers will be used for like structures.

For example, the wearable device 400 of the illustrated example has a housing 402 including the biometric monitoring system 106, the biometric sensor 124 including the light source 128 and the photo sensor 130, the biometric determiner 126, and the output 102 of the wearable device 100 of FIG. 1. Thus, the wearable device of the illustrated example employs a biometric monitoring system 106 that is substantially similar (e.g., identical) to the biometric monitoring system 106 of FIG. 1.

To enable functionality of the biometric monitoring system 106 of the wearable device 400 of FIG. 4, the wearable device 400 of the illustrated example includes a circuit 404. The circuit 404 of the illustrated example electrically couples and/or decouples the biometric monitoring system 106 and the power source 110 via a plurality of signal generators 406, a biometric system activator 408 and a switch operator 410.

The signal generators 406 of the illustrated example are communicatively coupled to the biometric system activator 408, and the biometric system activator 408 of the illustrated example is communicatively coupled to the switch operator 410. Based on one or more signals generated by the signal generators 406, the biometric system activator 408 commands the switch operator 410. For example, the biometric system activator provides a command output (e.g. a voltage, a current) to the switch operator 410. Based on the command output, the switch operator 410 of the illustrated example operates a switch 412 between a first or open position to electrically decouple (e.g., short-circuit) the power source 110 and the biometric monitoring system 106 (e.g., the light source 128) and a second or closed position to electrically couple the power source 110 and the biometric monitoring system 106 (e.g., the light source 128).

For example, FIG. 4 illustrates the circuit 404 in an open position and/or the power source 110 electrically decoupled from the biometric monitoring system 106. In particular, to electrically decouple the power source 110 and the biometric monitoring system, the switch 412 opens a grounding circuit 411 between the power source 110 and the biometric monitoring system 106. To electrically couple the power source 110 and the biometric monitoring system 106, the switch 412 closes the grounding circuit 411 between the power source 110 and the biometric monitoring system 106.

Each of the signal generators 406 of the illustrated example is similar (e.g., identical) to the switch 112 of FIG. 1. For example, each of the signal generators 406 of the illustrated example includes a spring contact 114 movable relative to the housing 402 of the wearable device 400 (e.g., spring-loaded buttons). More specifically, each spring contact 114 moves relative to the housing 402 between a first position 414 (e.g., an ON position) and a second position 416 (e.g., an OFF position).

A respective one of the signal generators 406 and/or the spring contact 114 is communicatively coupled (e.g., electrically coupled) to the biometric system activator 408 via a respective one of a conductive trace 418 (e.g., a dedicated conductive trace (e.g., a copper trace), a conductor, etc.). Thus, each of the signal generators 406 of the illustrated example have a dedicated communication path (e.g., the conductive trace 418) to communicate with the biometric system activator 408. In some examples, the conductive trace 418 may be a wire. In some examples, the conductive trace 418 may not be included. In some such examples, each of the signal generators 406 may be wirelessly coupled (e.g., via bluetooth communication, near-field communication protocol, etc.) to the biometric system activator 408.

Thus, each of the signal generators 406 generates a signal (e.g., a binary signal) representative of the respective one of the signal generators 406 being in the first position or the second position (e.g., on/off position). For example, when the spring contact 114 of a respective one of the signal generators 406 is in the first position 414, the spring contact 114 engages or electrically couples to the conductive trace 418 associated with the spring contact 114 (e.g., a dedicated trace coupling the spring contact 114 of the respective one of the signal generators 406). In turn, the respective one of the signal generators 406 in the first position provides a first instruction or first output signal 420 to the biometric system activator 408 via the conductive trace 418 associated with the respective one of the signal generators 406. The first output signal 420 of the illustrated example is representative of the spring contact 114 of the respective one of the signal generators 406 being in the first position 414. For example, when the spring contact 114 of a respective one of the signal generators 406 is in the second position 416, the spring contact 114 disengages or decouples from the corresponding conductive trace 418 (e.g., the dedicated trace coupling the spring contact 114 of the respective one of the signal generators 406). In turn, a respective one of the signal generators 406 in the second position provides a second instruction or second output signal 422 different than the first output signal 420 to the biometric system activator 408 via the conductive trace 418 associated with the respective one of the signal generators 406. The second output signal 422 of the illustrated example is representative of the spring contact 114 of the respective one of the signal generators 406 being in the second position 416.

In some examples, the signal generators 406 of the illustrated example may provide binary coded signals. For example, the first output signal 420 and the second output signal 422 may be binary signals. For example, the first output signal 420 may be a binary value of “1” and the second output signal 422 may be a binary value of “0”. In some examples, the first output signal 420 and the second output signal 422 may be any other type of signal or instruction.

The biometric system activator 408 of the illustrated example receives either the first output signal 420 or the second output signal 422 from each of the signal generators 406. In turn, the biometric system activator 408 of the illustrated example operates the switch operator 410 when the received first output signals 420 and/or the second output signals 422 satisfy a predetermined threshold (e.g., a combination of the first output signals 420 and/or the second output signals 422). For example, to enable the power source 110 to provide power (e.g., electrical current) to the biometric sensor 124 (e.g., the light source 128), the biometric system activator 408 of the illustrated example causes the switch operator 410 to move to a first or closed position to close the circuit 404 when a number of the first output signals 420 received by the biometric system activator 408 is greater than or equal to the predetermined threshold (e.g., or a number of received the second output signals 422 is less than the predetermined threshold). On the contrary, to open or short the circuit 404 so that the power source 110 is electrically decoupled from the biometric monitoring system 106 (e.g., the light source 128), the biometric system activator 408 of the illustrated example causes the switch operator 410 to move to a second or open position when a number of the first output signals 420 received by the biometric system activator 408 are less than the predetermined threshold (e.g., or a number of the received second output signals 422 is greater than or equal to the predetermined threshold).

The switch operator 410 of the illustrated example may be a transistor such as, for example, a metal oxide semiconductor field effect transistor (MOSFET), a negative metal oxide semiconductor (NMOS), a positive metal oxide semiconductor (PMOS), a complementary metal oxide semiconductor (CMOS) made from PMOS and NMOS transistors, a relay, a mechanical switch, and/or any other switch(es) or device(s) (e.g., electrical or mechanical) to electrically couple or decouple the power source 110 and the biometric monitoring system 106 based on a command output provided by the biometric system activator 408 that is determined by the first output signals 420 and/or the second output signals 422 generated by the signal generators 406.

To couple the wearable device 400 to a user, the housing 402 of the illustrated example includes a strap (e.g., a flexible or bendable strap) and a fastener or latch (e.g., a buckle). For example, the strap enables the housing 402 to couple (e.g., wrap) around a body portion of a user and the latch to secure the housing 402 to the user. In particular, a pressure or force imparted to (e.g., the spring contacts 114) of the signal generators 406 when the wearable device 400 is strapped and/or clamped to the body portion of the user causes the spring contact 114 of the signal generators 406 to move to the first position 414. In particular, a clamping force generated by the latching of the housing to the user's body portion is greater than a biasing force provided by the biasing element 122 urging the spring contact 114 toward the second position 416.

On the contrary, when the wearable device 400 is not coupled (e.g., clamped or secured) to a user's body portion (e.g., the latch is in an open position), the biasing element 122 restricts the spring contact 114 of the signal generators 406 from moving toward the first position 414. Thus, if the wearable device 400 is positioned on a flat surface and/or the latch of the wearable device 400 is in an open or unlatched position, the weight of the housing 402 and/or the wearable device 400 is not sufficient to cause the signal generators 406 (e.g., at least two or more signal generators 406) to move to the first position 414. As a result, if the wearable device 400 is positioned on a flat surface such that the signal generators 406 directly contact or engage the flat surface, the signal generators 406 (e.g., at least two or more of the signal generators 406) do not move to the first position 414 (e.g., remain in the second position 416 via the biasing element 122) and the circuit 404 remains in an open condition, thereby removing power to the biometric sensor 124 by electrically decoupling the power source 110 and the biometric sensor 124.

In some examples, the signal generators 406 of the illustrated example may be configured as electrical pressure switches. For example, the signal generators 406 may be pressure sensors, piezoelectric sensors, etc., that may generate a first signal (e.g., a first voltage or current) when the wearable device 400 is strapped to a user and a second signal (e.g., a second voltage or currant) different than the first signal when the wearable device 400 is removed from the user. In some such examples, the pressure sensor may be flush mounted relative to an outer surface of the housing 402.

FIG. 5 is a block diagram of the example biometric system activator 408 of FIG. 4. The biometric system activator 408 of the illustrated example of FIG. 4 may be implemented with logic gates, a logic circuit, a digital circuit, and/or other logic circuits or devices. However, in some examples, the biometric system activator 408 may be implemented with a processor executing instructions. The biometric system activator 408 of the illustrated example includes a signal receiver 502 and a switch activator 504 that are communicatively coupled via a bus 506.

The signal receiver 502 of the illustrated example receives the first output signal 420 from respective ones of the signal generators 406 when the respective ones of the signal generators 406 are in the first position, and the second output signal 422 from the respective ones of the signal generators 406 when the respective ones of the signal generators 406 are in the second position. For example, the signal receiver 502 is communicatively coupled to each of the conductive traces 418. In some examples, the signal receiver 502 includes a decoder to decode the first output signal 420 and the second output signal 422. For example, if the first output signal 420 or the second output signal 422 is provided as a voltage, a current, and/or other electrical measurement unit, the signal receiver 502 of the illustrated example may convert the first output signal 420 to a first binary value and the second output signal 422 to a second binary value.

As noted above, in some examples, the signal generators 406 of the illustrated example may be configured as pressure sensors, piezoelectric sensors, etc., that may generate a first voltage or current range when the wearable device 400 is strapped to a user and a second voltage or currant range different than the first voltage or current range when the wearable device 400 is removed from the user. In some such examples, the signal receiver 502 may decode the first voltage or current range by assigning it a first binary value and may decode the second voltage or current by assigning it a second binary value different than the first binary value. In some examples, the decoder may be implement by one or more logic gates. In some examples, the decoder may be implemented with instructions that are executed by a processor of the wearable device 400.

Based on the number of first output signals 420 and/or second output signals 422 received by the signal receiver 502, the switch activator 504 of the illustrated example determines whether to command the switch operator 410 to move the switch 412 to the open position or the closed position. More specifically, the switch activator 504 of the illustrated example determines if a number of the first output signals 420 and/or second output signals 422 from the signal generators 406 satisfies a predetermined threshold. For example, the switch activator 504 of the illustrated example is configured to detect when two or more of the signal generators 406 are in the first position 414 or if less than two signal generators 406 are in the second position 416 based on the received first output signals 420 and/or second output signals 422.

The switch activator 504 of the illustrated example may include a plurality of gate circuits or logic gates to determine if two or more of the signal generators 406 are in the first position 414 or the second position 416. In some examples, the logic device may include a signal aggregator to aggregate or sum up the values of the first output signals 420 and/or the second output signals 422 generated by the signal generators 406. For example, the first output signal 420 may be assigned a binary value of “1” and the second output signal 422 may be assigned a binary value of “0”. In some such examples, the signal aggregator aggregates or sums the binary values of the first output signal 420 or the second output signal 422 received or determined by the signal receiver 502. In some such examples, the switch activator 504 provides a command output to the switch operator 410 to cause the switch 412 to move to the closed position when the aggregate value is greater than a predetermined threshold (e.g., is greater than or equal to “2”). On the contrary, in some such examples, the switch activator 504 provides a command output to the switch operator 410 to cause the switch 412 to move to the open position when the aggregate value is less than the predetermined threshold (e.g., is less than “2”). In some such examples, the logic device may include a comparator to compare the number of first output signals 420 and/or second output signals 422 and/or the aggregate value to the predetermined threshold.

While an example manner of implementing the biometric system activator 406 of FIG. 4 is illustrated in FIG. 5, one or more of the elements, processes and/or devices illustrated in FIG. 5 may be combined, divided, re-arranged, omitted, eliminated and/or implemented in any other way. Further, the example signal receiver 502, the example switch activator 504 and/or, more generally, the example biometric system activator 406 of FIG. 4 may be implemented by hardware, software, firmware and/or any combination of hardware, software and/or firmware. Thus, for example, any of the example the example signal receiver 502, the example switch activator 5 and/or, more generally, the example biometric system activator 406 could be implemented by one or more analog or digital circuit(s), logic circuits, programmable processor(s), application specific integrated circuit(s) (ASIC(s)), programmable logic device(s) (PLD(s)), a field programmable gate device (FPGA(s)) and/or field programmable logic device(s) (FPLD(s)). When reading any of the apparatus or system claims of this patent to cover a purely software and/or firmware implementation, at least one of the example signal receiver 502, the example switch activator 5 and/or the example biometric system activator 406 is/are hereby expressly defined to include a non-transitory computer readable storage device or storage disk such as a memory, a digital versatile disk (DVD), a compact disk (CD), a Blu-ray disk, etc. including the software and/or firmware. Further still, the example biometric system activator 406 of FIG. 4 may include one or more elements, processes and/or devices in addition to, or instead of, those illustrated in FIG. 5, and/or may include more than one of any or all of the illustrated elements, processes and devices.

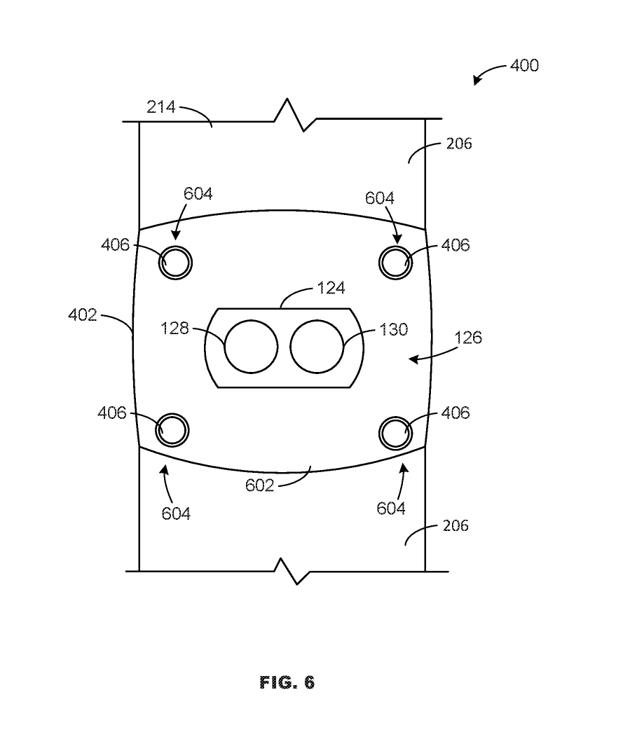

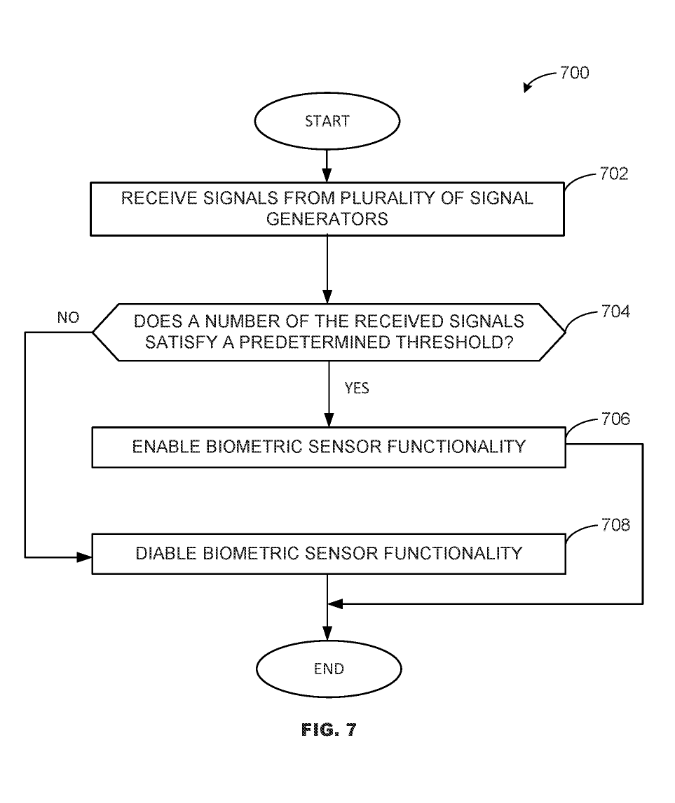

FIG. 6 illustrates an example implementation of the example wearable device 400 of FIGS. 4-5. The example wearable device 400 of the illustrated example may wrap around a user's wrist, arm, finger, torso, head, leg, ankle and/or any other body portion of the user. The housing 402 of the wearable device 400 of the illustrated example houses (e.g., encloses) the biometric monitoring system 106 (e.g., the biometric sensor 124 including the light source 128 and the photo sensor 130), the circuit 404, the switch 412, the biometric system activator 408, the switch operator 410, the power source 110 and the signal generators 406. The signal generators 406 and the biometric sensor 124 are positioned on a skin side 602 of the wearable device 400. In particular, the skin side 602 of the wearable device 400 is to engage a skin or body portion of a user when the wearable device 400 is strapped or coupled to the user.

The biometric sensor 124 of the illustrated example is positioned between the signal generators 406. More specifically, the signal generators 406 are spaced relative (e.g., about) a perimeter of the biometric sensor 124 and/or the housing 402. In particular, a respective one of the signal generators 406 of the illustrated example is positioned adjacent a corner 604 of the housing 402. In other examples, the signal generators 406 may be spaced relative to the skin side of the housing 402 in any suitable pattern. For example, the signal generators 406 may be positioned in a first row and a second row adjacent the first row. Additionally, the wearable device 400 of the illustrated example includes four signal generators. However, in some examples, the wearable device 400 may include more than four signal generators or less than four signal generators. In the illustrated example, the spring contacts 114 protrude from the skin side 602 of the housing 402 when the spring contacts 114 are in the second positions 416 and the spring contacts 114 are substantially flush relative to the skin side 602 when the spring contacts 114 are in the first positions 414.

The housing 402 couples to a user via a strap and a latch such as, for example, the strap 206 and the connector 212 of the wearable device 100 of FIGS. 2A-2C. In some examples, the plurality of signal generators 406 may be movably coupled to at least one of the housing 402 and/or a strap 206 of the housing 402. A force generated between the body portion of the user and the signal generators 406 when the housing 402 via the strap and the latch is secured or clamped to a body portion of the user causes the spring contacts 114 to move to the first position 414 against the force of the biasing element 122. Absent such clamping force, a spring force of the biasing element 122 restricts the spring contact 114 from moving to the first position 414. In other words, the circuit 404 remains in an open position unless the wearable device 400 is strapped or clamped to the body portion of the user. In examples in which the signal generators 406 are pressure sensors (e.g. electrical pressure sensors), the pressure sensors sense the clamping force generated when the wearable device 100 is strapped or coupled to the user. A force or pressure sensed when the housing 402 is positioned on a flat surface is significantly less than the clamping force generated via the strap and the latch when the wearable device 100 is strapped to a user. In some such examples, the pressure sensors may be flush mounted relative to the second side 602 of the housing 402.

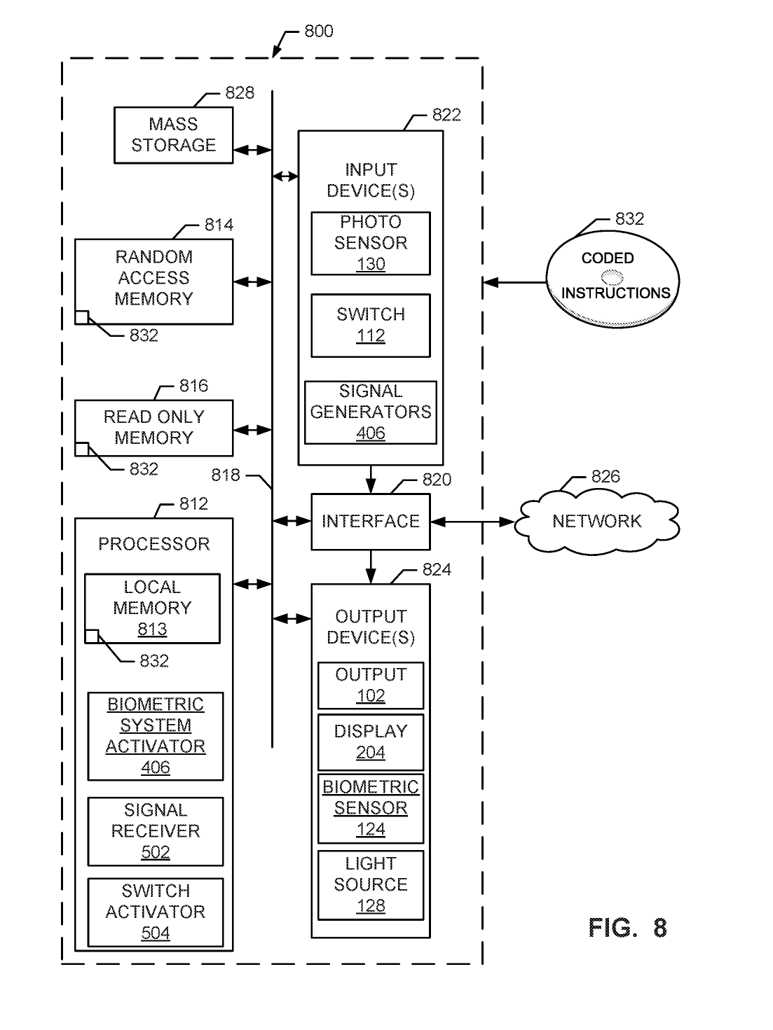

A flowchart representative of example machine readable instructions for implementing the biometric system activator 408 of FIG. 4 is shown in FIG. 7. In this example, the machine readable instructions comprise a program for execution by a processor such as the processor 812 shown in the example processor platform 800 discussed below in connection with FIG. 8. The program may be embodied in software stored on a non-transitory computer readable storage medium such as a CD-ROM, a floppy disk, a hard drive, a digital versatile disk (DVD), a Blu-ray disk, or a memory associated with the processor 812, but the entire program and/or parts thereof could alternatively be executed by a device other than the processor 812 and/or embodied in firmware or dedicated hardware. Further, although the example program is described with reference to the flowchart illustrated in FIG. 7, many other methods of implementing the example biometric system activator 408 may alternatively be used. For example, the order of execution of the blocks may be changed, and/or some of the blocks described may be changed, eliminated, or combined. Additionally or alternatively, any or all of the blocks may be implemented by one or more hardware circuits (e.g., discrete and/or integrated analog and/or digital circuitry, a Field Programmable Gate Array (FPGA), an Application Specific Integrated circuit (ASIC), a comparator, an operational-amplifier (op-amp), a logic circuit, etc.) structured to perform the corresponding operation without executing software or firmware.

As mentioned above, the example processes of FIG. 7 may be implemented using coded instructions (e.g., computer and/or machine readable instructions) stored on a non-transitory computer and/or machine readable medium such as a hard disk drive, a flash memory, a read-only memory, a compact disk, a digital versatile disk, a cache, a random-access memory and/or any other storage device or storage disk in which information is stored for any duration (e.g., for extended time periods, permanently, for brief instances, for temporarily buffering, and/or for caching of the information). As used herein, the term non-transitory computer readable medium is expressly defined to include any type of computer readable storage device and/or storage disk and to exclude propagating signals and to exclude transmission media. “Including” and “comprising” (and all forms and tenses thereof) are used herein to be open ended terms. Thus, whenever a claim lists anything following any form of “include” or “comprise” (e.g., comprises, includes, comprising, including, etc.), it is to be understood that additional elements, terms, etc. may be present without falling outside the scope of the corresponding claim. As used herein, when the phrase “at least” is used as the transition term in a preamble of a claim, it is open-ended in the same manner as the term “comprising” and “including” are open ended.

The program of FIG. 7 begins at block 702 when the biometric system activator 408 receives signals from each of the signal generators 406. For example, the signal receiver 502 of the example biometric system activator 408 receives either the first output signal 420 or the second output signal 422 from each of the signal generators 406.

The switch activator 504 determines if a number of the received signals satisfy a predetermined threshold (block 704). For example, the switch activator 504 may determine if more than two signal generators 406 are in the first position 414 based on the first output signals 420 and/or the second output signal 422 provided by the respective ones of the signal generators 406 and received by the signal receiver 502.

If the switch activator 504 determines that the number of signals received by the signal receiver 502 satisfy the predetermined threshold (block 704 is YES), the switch activator 504 enables biometric sensor functionality (block 706). For example, the switch activator 504 generates the command output to the switch operator 410 that cause the switch 412 (e.g., the circuit 404) to close when the switch activator 504 determines that the received signals are indicative of two or more of the signal generators 406 being in the first position 414 (e.g., or less than two of the signal generators 406 being in the second position 416).

If the switch activator 504 determines that the signals received by the signal receiver 502 from the signal generators 406 do not satisfy the predetermined threshold (block 704 is NO), the switch activator 504 disables biometric sensor functionality (block 708). For example, the switch activator 504 generates a command output to the switch operator 410 that causes the switch 412 (e.g., the circuit 404) to open when the switch activator 504 determines that the received signals are indicative of less than two of the signal generators 406 being in the first position 414 (e.g., or two or more of the signal generators 460 being in the second position 416).

FIG. 8 is a block diagram of an example processor platform 800 capable of executing the instructions of FIG. 7 to implement the biometric system activator 408 of FIGS. 4 and 5. The processor platform 800 can be, for example, a server, a personal computer, a mobile device (e.g., a cell phone, a smart phone, a tablet such as an iPad™), a personal digital assistant (PDA), an Internet appliance, or any other type of computing device.

The processor platform 800 of the illustrated example includes a processor 812. The processor 812 of the illustrated example is hardware. For example, the processor 812 can be implemented by one or more integrated circuits, logic circuits, microprocessors or controllers from any desired family or manufacturer. The hardware processor may be a semiconductor based (e.g., silicon based) device. In this example, the processor implements the biometric system activator 408, the switch operator 410, the signal receiver 502 and the switch activator 504.

The processor 812 of the illustrated example includes a local memory 813 (e.g., a cache). The processor 812 of the illustrated example is in communication with a main memory including a volatile memory 814 and a non-volatile memory 816 via a bus 818. The volatile memory 814 may be implemented by Synchronous Dynamic Random Access Memory (SDRAM), Dynamic Random Access Memory (DRAM), RAMBUS Dynamic Random Access Memory (RDRAM) and/or any other type of random access memory device. The non-volatile memory 816 may be implemented by flash memory and/or any other desired type of memory device. Access to the main memory 814, 816 is controlled by a memory controller.

The processor platform 800 of the illustrated example also includes an interface circuit 820. The interface circuit 820 may be implemented by any type of interface standard, such as an Ethernet interface, a universal serial bus (USB), and/or a PCI express interface.

In the illustrated example, one or more input devices 822 are connected to the interface circuit 820. The input device(s) 822 permit(s) a user to enter data and/or commands into the processor 812. The input device(s) can be implemented by, for example, an audio sensor, a microphone, a camera (still or video), a keyboard, a button, a mouse, a touchscreen, a track-pad, a trackball, isopoint and/or a voice recognition system.

One or more output devices 824 are also connected to the interface circuit 820 of the illustrated example. The output devices 824 can be implemented, for example, by display devices (e.g., a light emitting diode (LED), an organic light emitting diode (OLED), a liquid crystal display, a cathode ray tube display (CRT), a touchscreen, a tactile output device, a printer and/or speakers). The interface circuit 820 of the illustrated example, thus, typically includes a graphics driver card, a graphics driver chip and/or a graphics driver processor.

The interface circuit 820 of the illustrated example also includes a communication device such as a transmitter, a receiver, a transceiver, a modem and/or network interface card to facilitate exchange of data with external machines (e.g., computing devices of any kind) via a network 826 (e.g., an Ethernet connection, a digital subscriber line (DSL), a telephone line, coaxial cable, a cellular telephone system, etc.).

The processor platform 800 of the illustrated example also includes one or more mass storage devices 828 for storing software and/or data. Examples of such mass storage devices 828 include floppy disk drives, hard drive disks, compact disk drives, Blu-ray disk drives, RAID systems, and digital versatile disk (DVD) drives.

The coded instructions 832 of FIG. 7 may be stored in the mass storage device 828, in the volatile memory 814, in the non-volatile memory 816, and/or on a removable tangible computer readable storage medium such as a CD or DVD.

Example wearable devices are disclosed. Further examples and combinations thereof include the following.

Example 1 may be a wearable device including a housing having a biometric sensor to detect physiological information of a user. The wearable device includes a circuit to electrically couple the biometric sensor and a power source of the wearable device; and a spring contact positioned adjacent the biometric sensor and to be oriented toward a user, the spring contact being movably coupled relative to the housing. The spring contact is to close the circuit to activate the biometric sensor when the wearable device is strapped to the user and the spring contact is in engagement with the user. The spring contact is to open the circuit to deactivate the biometric sensor when the wearable device is removed from the user to reduce energy drain of the power source by the biometric sensor.

Example 2 may include the subject matter of example 1, wherein the spring contact includes a button and a spring positioned in a cavity of the housing, the button to project from an outer surface of the housing when the wearable device is not strapped to the user.

Example 3 may include the subject matter of any one of examples 1 or 2, wherein the biometric sensor includes an optical heart rate monitor having a light emitting diode and a photo sensor.

Example 4 may include the subject matter of any one of examples 1-3, wherein the spring contact is to electrically couple the power source and the light emitting diode when the wearable device is strapped to the user and the spring contact is to electrically decouple the power source and the light emitting diode when the wearable device is not strapped to the user.

Example 5 may include the subject matter of any one of examples 1-4, wherein the light emitting diode is positioned in a surface of the button.

Example 6 may be a wearable device including a housing having a biometric monitoring system to detect a physiological characteristic of a user. The housing defines a first side and a second side opposite the first side. The first side of the housing to engage a body of the user when the housing is strapped to the user. A switch is movably coupled to the first side of the housing. The switch is to move between a first position and a second position relative to the first side of the housing. In the first position, the switch is to enable biometric monitoring functionality. In the second position, the switch is to disable biometric monitoring functionality. The switch is to move to the first position when the housing is strapped to the user. The switch is to protrude from the first side of the housing when the switch is in the second position.

Example 7 may include the subject matter of example 6, wherein the switch is to electrically couple a power source and the biometric monitoring system when the switch is in the first position, and the switch is to electrically decouple the power source and the biometric monitoring system when the switch is in the second position.

Example 8 may include the subject matter of any one of examples 6 or 7, wherein the switch includes a spring contact movable relative to the housing, and a biasing element to urge the spring contact toward the second position.

Example 9 may include the subject matter of any one of examples 6-8, wherein the biasing element is to allow the spring contact to move to the first position only when the housing is strapped to the body of the user.

Example 10 may include the subject matter of any one of examples 6-9, wherein the biasing element is to restrict the spring contact from moving to the first position when the housing is not strapped to the body of the user.

Example 11 may include the subject matter of any one of examples 6-10, wherein the biometric monitoring system includes an optical heart rate monitor.

Example 12 may include the subject matter of any one of examples 6-11, wherein the optical heart rate monitor includes a light emitting diode and a photo sensor.

Example 13 may include the subject matter of any one of examples 6-12, wherein the spring contact interrupts a conductive trace between a power source and the light emitting diode of the biometric monitoring system when the spring contact is in the second position.

Example 14 may include the subject matter of any one of examples 6-13, wherein at least one of the light emitting diode or the photo sensor is positioned in a body of the spring contact.

Example 15 may be a wearable device including a housing having a biometric sensor and a power source. A strap coupled to the housing and positionable between a closed position to clamp the housing against a body of a user and an open position to release the housing from the body of the user. A plurality of signal generators movably coupled to at least one of the housing or the strap and movable relative to the at least one of the housing or the strap between a first position and a second position, the signal generators to engage the body of the user when the housing is coupled to the user. A logic device is to receive signals from the signal generators. The logic device to electrically couple the power source and the biometric sensor when a number of received signals indicative of the signal generators being in the first position is greater than a threshold, and electrically decouple the power source and the biometric sensor when the number of received signals indicative of the signal generators being in the second position is less than the threshold.

Example 16 may include the subject matter of example 15, wherein the biometric sensor includes an optical heart rate monitor having a light emitting diode and a photo sensor.

Example 17 may include the subject matter of any one of examples 15 or 16, wherein the logic device further includes a transistor to receive a command from the logic device to operate a switch between an open position when the number of received signals is less than the threshold and a closed position when the number of received signals is greater than the threshold, the switch in the open position to electrically decouple the power source and the biometric sensor and the switch in the closed position to electrically couple the power source and the biometric sensor.

Example 18 may include the subject matter of any one of examples 15-17, wherein the housing defines a first side and a second side opposite the first side, the first side of the housing to engage a body of the user when the housing is strapped to the user.

Example 19 may include the subject matter of any one of examples 15-18, wherein the plurality of signal generators includes spring contacts positioned to protrude from the first side of the housing.

Example 20 may include the subject matter of any one of examples 15-19, wherein spring contacts are positioned around a perimeter of the biometric sensor.

Example 21 includes a method for controlling a biometric sensor of a wearable device, the method including receiving signals from a plurality of signal generators positioned on a housing that has a biometric sensor; determining if a number of received signals is greater than a threshold; enabling biometric sensor functionality when the number of received signals is greater than the threshold; and disabling biometric sensor functionality when the number of received signals is less than the threshold.

Example 22 includes the method of example 21, wherein the enabling biometric sensor functionality includes electrically coupling the biometric sensor to a power source via a switch.

Example 23 includes the method of at least one of examples 21 or 22, wherein the disabling biometric sensor functionality includes electrically decoupling the biometric sensor from a power source via the switch.

Example 24 includes a tangible computer-readable medium comprising instructions that, when executed, cause a processor to, at least: receive signals from a plurality of signal generators positioned on a housing that has a biometric sensor; determine if a number of received signals is greater than a threshold; enable biometric sensor functionality when the number of received signals is greater than the threshold; and disable biometric sensor functionality when the number of received signals is less than the threshold.

Example 25 may include the computer-readable medium as defined in example 24, wherein when executed, further cause the machine to electrically couple the biometric sensor to a power source to enable the biometric sensor functionality.

Example 26 may include the computer-readable medium as defined in examples 24 or 25, wherein when executed, further cause the machine to electrically decouple the biometric sensor from a power source to disable the biometric sensor functionality.

Example 27 may be a wearable device including a housing including means for sensing physiological information of a user; means for clamping the sensing means to a body of the user; and means for activating the sensing means, the activating means being movably coupled to at least one of the housing or the clamping means, the activating means to cause the sensing means to draw power from a power source positioned in the housing when the sensing means is strapped to the body of the user via the clamping means, the activating means to restrict the sensing means from drawing power from the power source when the sensing means is not clamped against the body of the user.

Example 28 may include the subject matter of example 27, wherein the activating means includes means for electrically coupling and electrically decoupling the power source and the sensing means.

Although certain example methods, apparatus and articles of manufacture have been disclosed herein, the scope of coverage of this patent is not limited thereto. On the contrary, this patent covers all methods, apparatus and articles of manufacture fairly falling within the scope of the claims of this patent.

What is claimed is:

1. A wearable device comprising
a housing including a biometric sensor to detect physiological information of a user;
a circuit to electrically couple the biometric sensor and a power source of the wearable device; and
a spring contact positioned adjacent the biometric sensor and to be oriented toward a user, the spring contact being movably coupled relative to the housing, the spring contact is to close the circuit to activate the biometric sensor when the wearable device is strapped to the user and the spring contact is in engagement with the user, the spring contact is to open the circuit to deactivate the biometric sensor when the wearable device is removed from the user to reduce energy drain of the power source by the biometric sensor.

2. The device of claim 1, wherein the spring contact includes a button and a spring positioned in a cavity of the housing, the button to project from an outer surface of the housing when the wearable device is not strapped to the user.

3. The device of claim 2, wherein the biometric sensor includes an optical heart rate monitor having a light emitting diode and a photo sensor.

4. The device of claim 3, wherein the spring contact is to electrically couple the power source and the light emitting diode when the wearable device is strapped to the user and the spring contact is to electrically decouple the power source and the light emitting diode when the wearable device is not strapped to the user.

5. The device of claim 3, wherein the light emitting diode is positioned in a surface of the button.

6. A wearable device comprising:
a housing having a biometric monitoring system to detect a physiological characteristic of a user, the housing defining a first side and a second side opposite the first side, the first side of the housing to engage a body of the user when the housing is strapped to the user; and
a switch movably coupled to the first side of the housing, the switch to move between a first position and a second position relative to the first side of the housing, in the first position, the switch is to enable biometric monitoring functionality, and in the second position, the switch is to disable biometric monitoring functionality, the switch to move to the first position when the housing is strapped to the user, the switch to protrude from the first side of the housing when the switch is in the second position.

7. The device of claim 6, wherein the switch is to electrically couple a power source and the biometric monitoring system when the switch is in the first position, and the switch is to electrically decouple the power source and the biometric monitoring system when the switch is in the second position.

8. The device of claim 6, wherein the switch includes a spring contact movable relative to the housing, and a biasing element to urge the spring contact toward the second position.

9. The device of claim 8, wherein the biasing element is to allow the spring contact to move to the first position only when the housing is strapped to the body of the user.

10. The device of claim 8, wherein the biasing element is to restrict the spring contact from moving to the first position when the housing is not strapped to the body of the user.

11. The device of claim 8, wherein the biometric monitoring system includes an optical heart rate monitor.

12. The device of claim 11, wherein the optical heart rate monitor includes a light emitting diode and a photo sensor.

13. The device of claim 12, wherein the spring contact interrupts a conductive trace between a power source and the light emitting diode of the biometric monitoring system when the spring contact is in the second position.

14. The device of claim 13, wherein at least one of the light emitting diode or the photo sensor is positioned in a body of the spring contact.

15. A wearable device comprising:
a housing having a biometric sensor and a power source;
a strap coupled to the housing and positionable between a closed position to clamp the housing against a body of a user and an open position to release the housing from the body of the user;
a plurality of signal generators movably coupled to at least one of the housing or the strap and movable relative to the at least one of the housing or the strap between a first position and a second position, the signal generators to engage the body of the user when the housing is coupled to the user; and
a logic device to receive signals from the signal generators, the logic device to electrically couple the power source and the biometric sensor when a number of received signals indicative of the signal generators being in the first position is greater than a threshold, and electrically decouple the power source and the biometric sensor when the number of received signals indicative of the signal generators being in the second position is less than the threshold.

16. The device of claim 15, wherein the biometric sensor includes an optical heart rate monitor having a light emitting diode and a photo sensor.

17. The device of claim 15, wherein the logic device further includes a transistor to receive a command from the logic device to operate a switch between an open position when the number of received signals is less than the threshold and a closed position when the number of received signals is greater than the threshold, the switch in the open position to electrically decouple the power source and the biometric sensor and the switch in the closed position to electrically couple the power source and the biometric sensor.

18. The device of claim 15, wherein the housing defines a first side and a second side opposite the first side, the first side of the housing to engage a body of the user when the housing is strapped to the user.

19. The device of claim 18, wherein the plurality of signal generators includes spring contacts positioned to protrude from the first side of the housing.

20. The device of claim 19, wherein spring contacts are positioned around a perimeter of the biometric sensor.
